(19) United States
(12) Patent Application Publication
(10) Pub. No.: US 2017/0151398 A1
(43) Pub. Date: Jun. 1, 2017

(54) SYSTEMS WITH CONTROL MECHANISM FOR NEGATIVE PRESSURE AND POSITIVE PRESSURE FOR OPTIMIZATION OF VENTILATION, CENTRAL HEMODYNAMICS, AND VITAL ORGAN PERFUSION

(71) Applicants: Kevin R. WARD, Superior Township, MI (US); James M. BLUM, Ann Arbor, MI (US); Kayvan NAJARIAN, Northville, MI (US)

(72) Inventors: Kevin R. WARD, Superior Township, MI (US); James M. BLUM, Ann Arbor, MI (US); Kayvan NAJARIAN, Northville, MI (US)

(73) Assignee: THE REGENTS OF THE UNIVERSITY OF MICHIGAN, Ann Arbor, MI (US)

(21) Appl. No.: 15/316,409

(22) PCT Filed: Jun. 15, 2015
(86) PCT No.: PCT/US2015/035731

§ 371 Date: Dec. 5, 2016

Related U.S. Application Data
(60) Provisional application No. 62/011,912, filed on Jun. 13, 2014.

(51) Int. Cl. A61M 16/00; A61B 5/021; A61B 5/0205; A61B 17/135; A61B 5/03

(52) U.S. Cl. CPC A61M 16/0009; A61M 16/0051; A61M 16/0069; A61B 17/1355; A61B 5/031; A61B 5/0205; A61B 5/021; A61M 2205/3344; A61M 2205/3303; A61M 2205/52; A61M 2205/502; A61M 2230/04; A61M 2230/205; A61M 2230/65; A61M 2202/0488; A61M 2230/40

(57) ABSTRACT

An intelligent control system for controlling a patient ventilation apparatus having at least negative end expiratory pressure ventilation (NEEP) system. The controller accepts signals from one or more physiological parameter sensors, compares data received from those sensors to target values stored in a memory, and either recommends changes to the NEEP or signals the NEEP to change or maintain certain settings, such as level and frequency. A positive end pressure ventilation system may be further employed with the intelligent control system, such that, based on data received from the physiological sensors, the intelligent control system can be used to deliver optimal levels of negative pressure ventilation and positive pressure ventilation, and promote weaning of a patient off the ventilators. The intelligent control system may also be used with an external pressure oscillatory device.

### REFERENCE TO RELATED APPLICATIONS

This is a non-provisional of, and claims the benefit of the filing date of, U.S. Provisional Application No. 62/011,912, filed Jun. 13, 2014, the entirety of which is hereby incorporated by reference.

### FIELD OF THE DISCLOSURE

The present disclosure relates generally to respiratory ventilation systems and, more specifically, to the regulation and optimization of negative pressure ventilation and positive pressure ventilation in patient respiratory ventilation systems which in turn are used to optimize ventilation, central hemodynamics, and vital organ perfusion.

### BACKGROUND

Ventilators are machines designed to mechanically move breathable air into and out of the lungs, to provide the mechanism of breathing for a patient who is physically unable to breathe, or breathing insufficiently. Monitoring various physiological parameters provides raw data to clinicians to help them assess how therapy is proceeding. However, these parameters tell them very little about how to adjust the ventilator settings. Clinicians regularly monitor patients' ability to cope with a given support level and then decide whether support should be further reduced, maintained or increased based on their experience and knowledge. An intelligent system which is aimed to assist, rather than replace, the clinician would therefore result in improved patient comfort and safety. Intelligent systems can also play a vital role in weaning patients from ventilators. Currently, weaning tends to be undertaken in a somewhat ad hoc manner, and the clinician, once more, must rely on his or her experience and knowledge. However, there is evidence that weaning may proceed more efficiently if directed according to some specified protocol.

Negative pressure ventilation (NPV) was once the primary mode of assisted mechanical ventilation in the setting of respiratory failure. However, during the Copenhagen polio epidemic in the 1950's when insufficient resources were available, the use of positive pressure ventilation through an endotracheal tube was necessary. Since that time, NPV has fallen out of favor and is now used only in niche markets of cystic fibrosis, chest wall deformities, and other relatively rare disorders, which make long term normal ventilation difficult. In these settings, NPV has been utilized using a chest curiass. A common NPV device of these is the Hayek RTX respirator device, available from United Hayek Industries (Medical) of London, UK, which employs what Hayek refers to a biphasic cuirass ventilation technique.

Despite the dominance of positive pressure mechanical ventilation (PPV) in the field of critical care, PPV is not without problems. PPV produces physiology opposite of the intrathoracic negative pressure mechanism that humans use to breath normally. The elevated intrathoracic and lung pressure that PPV produces can result in lung injury through excessive stretch of lung tissue. In addition, elevated intrathoracic pressure can result in decreases in venous return and other consequences which can worsen central cardiovascular hemodynamics and intracranial pressure. This can result in decreases in vital organ perfusion, for example to the brain and splanchnic bed.

Given little progress in the treatment of critical illness and injury ranging from the treatment of acute lung injury, respiratory failure, cardiogenic shock, sepsis, traumatic brain injury, and others, there is a need to re-engineer NPV and other negative pressure modalities to improve the effectiveness of mechanical ventilation, improve central and peripheral hemodynamics and perfusion and improve cerebral physiology. Such methods would also greatly improve therapeutic drug delivery to the lung.

In order to better leverage NPV and other negative pressure modalities (external negative abdominal and external negative neck pressure), control and feedback mechanisms will be necessary so that these negative pressure modalities work in concert with both current invasive and noninvasive mechanical PPV methods as well as various invasive and noninvasive monitoring technologies of the cardiopulmonary, cerebrovascular, and peripheral vascular and organ systems.

### SUMMARY OF THE DISCLOSURE

The present disclosure includes an intelligent monitoring/controller system that interfaces with a ventilator and patient, to optimize the use of negative pressure ventilation (NPV) and its various parameters such as negative end expiratory pressure ventilation (NEEP) modalities, as well as positive pressure ventilation (PPV) including its parameters such as positive end expiratory pressure (PEEP) modalities. The intelligent monitoring/controller system may manipulate the rate, frequency, and force of these measures to achieve desired endpoints.

The system of the present disclosure uses physiological signals specific to a disease or condition of the patient, such as such as esophageal pressure, intracranial pressure, intraabdominal pressure, bladder pressure, arterial blood pressures, central venous and pulmonary artery pressure, arterial and venous oxygenation, tissue oxygenation, electrical activity of the heart (as is measured by an electrocardiogram), impedance, cerebral blood flow, cardiac output, stroke volume variation, pulse pressure variation, and others, to determine how ventilation should be optimized. Using machine learning methods, the system then determines, based on the physiological signals, what kind of ventilation is best suited to the particular condition, when to stop ventilation, when to resume ventilation, and facilitates gradually weaning the patient off the ventilator so that the patient may resume normal breathing.

The control system of the present disclosure optimizes the use of negative pressure modalities, alone or together with for a variety of clinical applications. The control system of the present disclosure also optimizes treatment of acute lung injury, acute respiratory failure, cardiogenic and other shock states, traumatic brain injury and other cerebrovascular catastrophes. The system can decrease incidence of abdominal compartment syndrome, enhances weaning from mechanical ventilation and resumption of normal breathing, and can be used to improve delivery of therapeutic agents to the lung.

The patient monitoring data collected by the control system of the present disclosure may be easily integrated with electronic medical records. The control system may also be configured to interface with communication systems so as to deliver alerts to medical professionals, thereby enabling the transmission of ventilator alarms to a clinician's pager, computer monitor, tablet, public address system, two-way radio, watch, and/or smartphone device to enable fast response without clinician being in earshot of ventilator or patient.

The system is capable of leveraging other negative pressure devices such as a negative pressure collar capable of producing fluctuating or continuous levels of positive or negative pressure, which can be helpful for relieving intracranial pressure following, for example, a traumatic brain injury. Abdominal curiass's which either span the abdomen or abdomen and chest are also envisioned to have utility with the system allowing for production of continuous levels of positive or negative pressure working with the system in conjunction with the aforementioned ventilation systems and physiologic signals to optimize ventilation as well as central hemodynamics and vital organ perfusion.

By taking data from nonlinear sources, namely sensor data indicative of physiologic parameters such as cardiac output, tissue oxygenation, lung pressures, arterial blood pressure (ABP), cerebral pressure perfusion, esophageal pressure, intraabdominal pressure, bladder pressure, arterial blood pressures, central venous pressure, pulmonary artery pressure, arterial oxygenation, venous oxygenation, tissue oxygenation, electric activity of the heart, impedance, cerebral blood flow, cardiac output, stroke volume variation, intracranial pressure, and pulse pressure variation, and based on that data, controlling the delivery of positive or negative pressure, therapeutic results can be achieved with great precision and efficiency. A controller that is programmed to make certain adjustments or recommendations for adjustments to positive and negative pressure delivery based on physiologic parameter data essentially works in concert with the body's cardiovascular and neurological systems.

Examples of the manner in which the system of the present disclosure may be configured are shown schematically in the drawings, as described below.

### BRIEF DESCRIPTION OF THE SEVERAL VIEWS OF THE DRAWINGS

FIG. 1 is a schematic diagram of a first embodiment of a control system of the present disclosure, in which negative end expiratory pressure ventilation (NEEP) and positive end expiratory pressure ventilation (PEEP) are employed simultaneously, and the control system is configured to ensure an optimal setting for each of the NEEP and PEEP devices at any given time based on a patient's physiological data. The control system may be used for oscillatory negative and positive pressure ventilation as well;

FIG. 2 is a schematic diagram of a second embodiment of a control system of the present disclosure, in which only NEEP is utilized, and NEEP may be used for oscillatory negative pressure, constant negative pressure, or both; and

FIG. 3 is a schematic diagram of a third embodiment of a control system of the present disclosure, in which external pressure oscillatory devices such as a neck collar or abdominal curiass is used either alone or in conjunction with positive pressure and negative pressure ventilation to further effect and optimize ventilatory, hemodynamics, or vital organ perfusion response.

### DETAILED DESCRIPTION OF THE PREFERRED EMBODIMENTS

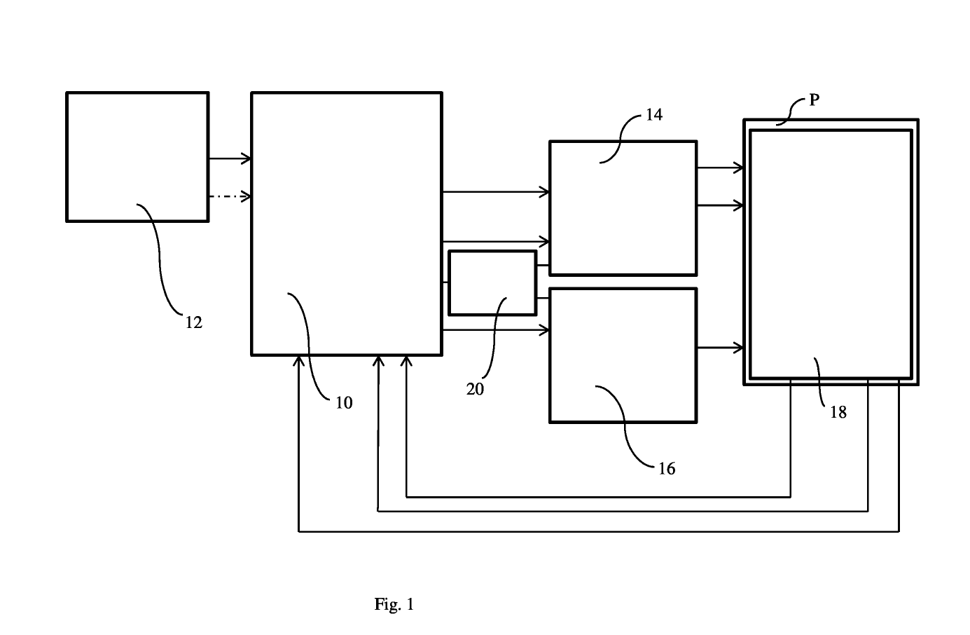

With reference to FIG. 1, a controller in the form of an intelligent control system 10 of the present disclosure for use in controlling ventilation systems receives a set of desired or target values for the physiological signals of interests, which may be provided according to a database that is stored in a memory 12 of the intelligent control system 10. The memory 12 may be a local memory, or, as indicated by the dash-dotted line between the intelligent control system and the memory 12 in FIG. 1, the memory 12 may be provided or updated remotely, enabling the set of desired or target values to be updated from time to time in accordance with, for instance, updated research as to best practices for target values for various physiological parameters as indicators for stages or goals in a patient's ventilation therapy.

The intelligent control system 10 of this first embodiment is in controlling communication with a positive end expiratory pressure ventilation (PEEP) system 14 and a negative end expiratory pressure ventilation (NEEP) system 16, which are in communication with a breathing tube for a patient P. One or more patient physiological parameter measuring devices 18 that are in communication with at least the intelligent control system 10 are applied to the patient P, and provide the intelligent control system 10 with physiological signals specific to a disease or condition of the patient, such as such as esophageal, intracranial, arterial blood pressures, oxygenation, and/or blood flow.

The intelligent control system 10 is in further communication with a logic control that constantly, or at periodic intervals, compares one or more of the physiological signals received from the patient physiological parameter measuring devices 18 to a desired or target value for the physiological parameter associated with that that one or more physiological signal from the memory 12, and sends appropriate control signals to the NEEP 16 and/or PEEP 14 based on those comparisons in accordance with the programmed logic control. The intelligent control system 10 may be programmed to make certain adjustments to the NEEP and/or PEEP automatically based on the comparisons, and with regard to other adjustments, not implement the adjustment until after a recommendation has been sent to a clinician, such as via a display 20, and that clinician has approved the adjustment to be made. The intelligent control system 10 may be further programmed to accept a manually-entered command to effect an adjustment to one or more of the NEEP 14 and PEEP 16 ventilators other than that which is recommended from the programmed logic control. The display 20 may be in communication with the intelligent control system 10, as well as neither, one, or both of the NEEP 14 and the PEEP 16.

In case of adult respiratory distress syndrome, it is often the case that the objective of ventilation is to achieve and maintain a certain desired level for esophageal pressure, which in this control scheme is referred to as r_{d}. Considering r_{d} and the actual values of all relevant measured/estimated physiological signals, the intelligent control system 10 first decides whether to use only PEEP 14, only NEEP 16, or some combination of the two for ventilation. In other words, at any time, by analyzing the physiological signals the intelligent control system 10 will decide to operate in one of the following four modes of operations:

I. Constant NEEP without PEEP: The intelligent control system 10 can recommend, based on the analysis of one or more physiological signals received from the patient physiological parameter measuring devices 18, and comparison of the physiological signals to a desired or target value for the physiological parameter associated with that that one or more physiological signal from the memory 12, that it would be beneficial to only use a contact NEEP and remove PEEP. In this case, the intelligent control system 10 will calculate the optimal level of negative pressure within any given time window, signal the NEEP 14 to deliver that level of negative pressure, and update the calculation based on patient's physiological changes.

II. Constant NEEP with oscillating PEEP: In this mode, in each time window, the intelligent control system 10 will calculate the optimal values for the constant level of NEEP, the frequency of PEEP as well as the level (magnitude) of PEEP.

Note that the possible mode of applying PEEP without constant NEEP is equivalent to having PEEP with almost zero level of constant NEEP which is a special case of mode II and is automatically generated by the intelligent control system 10. Another function of the intelligent control system 10 is to recommend which mode of operation discussed above is needed, or if the entire ventilation is no longer needed.

Specifically, a task of the controller is to form a recommendation (to care givers), based on the analysis of physiological signals at previous time windows, to gradually stop ventilation and allow patient to breathe normally. The controller will also recommend if ventilation, at a specific mode of operation, has to be resumed/started.

In each of the above mentioned modes of operation, the patient P receives PEEP and/or NEEP ventilation with the optimal levels and frequency at any given time. Physiological parameter measuring devices 18 measure all relevant physiological signals which are provided to the intelligent control system 10 for decision making and control analysis. For instance, in case of ARDS, these signals can include: esophageal pressure (the signal to be controlled), intrathoracic pressure and inspiratory pressure.

Structure 2:

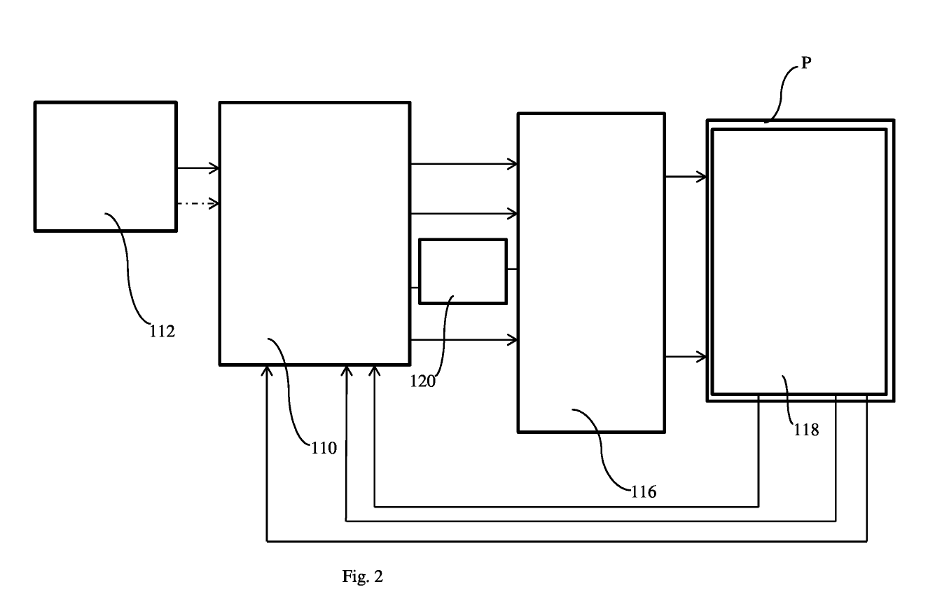

The second structure, shown in FIG. 2, deals with applications where only NEEP 116 is available or used. In such cases, NEEP can be used for oscillatory negative pressure, constant negative pressure or both. The system would also allow the curiass producing NEEP to produce positive pressure to assist with expiration in an active manner to ensure more complete along the emptying which may be necessary in such disease states as asthma which produce bronchospasm. As in the arrangement illustrated in FIG. 1 and discussed above, the intelligent control system 110 of this embodiment receives a desired level for the targeted physiological signals from a memory 112, as well as the actual values of these and other physiological signals which are continuously monitored by physiological parameter measuring devices 118 in communication with the intelligent control system 110.

As example of this in treatment of traumatic brain injury patients (TBI) is controlling the level of intracranial pressure, ICP, at or below a certain level. In such an application, ICP as well as other signals including arterial blood pressure (ABP) and cerebral pressure perfusion are measured and provided to the intelligent control system 110. Values associated with one or more of these physiological parameter signals are compared by the intelligent control system 110 to a respective desired or target value for the physiological parameter from the memory 112, and the intelligent control system 110 sends appropriate control signals to the NEEP 116 based on those comparisons in accordance with the programmed logic control, or sends a recommendation, such as to a display 120 in communication with the intelligent control system 110, to make an appropriate adjustment to, or maintain a particular level and/or frequency of, the negative pressure delivered by the NEEP. For instance, based on the comparison of signals received from each of the one or more physiological parameter sensors to a respective one of the target values included in the memory, the controller generates one of a recommendation that the NPV should be set to deliver an oscillatory magnitude and a recommendation that the NPV should be set to deliver an oscillatory frequency of negative pressures. In this manner, NPV is utilized effectively for therapeutic effects. NPV is controlled in a manner that enhances cerebral blood flow. Use of such a system would bring even greater value to the victim of multisystem trauma. Multisystem trauma patients regularly have multiple injuries including TBI, lung injury and cardiovascular shock. By using the system described above, the enhancement of cerebral blood flow can occur simultaneously to reducing PPV inspiratory pressures that might otherwise be injurious to the lung. Increases in cerebral blood occur as a result of both reductions in intracranial pressure and also enhancements of cardiac output through the actions of NPV increasing venous return to the heart, all while countering the injurious effects of PPV on lung injury.

Another example would be to utilize the system as a countermeasure to patients who have septic shock and adult respiratory distress syndrome (ARDS). ARDS can be associated with significant decreases in lung compliance where lung injury is exacerbated by increasing PPV but where increases in PPV via PEEP may be needed to provide adequate oxygenation. This scenario is further complicated by the fact that sepsis can result in myocardial depression and reductions in cardiac output which exacerbate systemic tissue hypoxia. Use of PPV can further reduce cardiac output and tissue blood flow. The use of the system described previously would greatly improve the physiology of such a complex patient. Through incorporation of physiologic signals such as arterial blood pressure, cardiac output, airway pressure, tissue oxygenation, central venous pressure, tissue impedance, and others, the optimal combination of NPV and PPV can be found which optimizes cardiac output and tissue oxygenation while simultaneously enhancing arterial blood oxygen and carbon dioxide at the lowest airway pressure. Continuous use of such as system becomes extremely critical during the transition of patients off the use of vasopressors and as weaning from mechanical ventilation occurs.

As in Structure 1, in the structure of this embodiment, the intelligent control system 110 can function in one of the following functional modes:

I. Constant NEEP without oscillating NEEP: The operation of the intelligent control system 110 to achieve this mode would be the same as mode I of Structure 1.

II. Constant NEEP with oscillating NEEP: In this mode, in each time window, the intelligent control system 110 will calculate the optimal values for the constant level of NEEP, the frequency of NEEP as well as the level (magnitude) of NEEP.

Again note that the possible mode of applying oscillating NEEP without constant NEEP is equivalent to having NEEP with almost zero level of constant NEEP which is a special case of mode II and is automatically generated by the intelligent control system 110.

Again, also note that the potential for application of other external negative and positive pressure devices such as a neck collar or abdominal curiass is anticipated to be used with or without the systems above to improve ventilation, central hemodynamics and vital organ perfusion. While NPV can be controlled to enhance cerebral blood flow, for example, PPV can be controlled to modulate cerebral blood flow, aiding in therapy for traumatic brain injury patients.

In addition, as in Structure 1, another function of the intelligent control system 110 is to recommend which mode of operation discussed above is needed, or if the entire ventilation is no longer needed. Or if manipulation of central hemodynamics or vital organ perfusion is no longer needed. Specifically, a task of the intelligent control system 110 is to form a recommendation (to care givers), based on the analysis of physiological signals at previous time windows, to gradually stop ventilation and allow patient to breathe normally. The intelligent control system 110 will also recommend if ventilation, at a specific mode of operation, has to be resumed/started.

The systems of the present disclosure are intended to be used in constant or oscillating conditions of combined positive pressure ventilation and negative pressure ventilation to improve lung function, reduce lung injury associated with PPV, enhance central and peripheral hemodynamics and organ function, improve cerebral perfusion pressure, improve delivery of therapeutic agents to the lung given via inhalation methods, and assist in weaning from mechanical ventilation. A significant example of this would be to use the combination of NPV and PPV to enhance the delivery and distribution of such lung therapeutic agents as surfactants to injured lung areas. Delivery of surfactants has only been done via PPV which has not been demonstrated to be helpful due to the probability that natural negative inspiratory pressure cannot be leveraged in this mode. Through the use of surfactants coupled with NPV, enhanced distribution of surfactants coupled with the PPV alleviating effects of NPV on lung tissue, reductions in acute lung injury resulting in improved oxygenation and ventilation can occur. This same approach could be used for improved delivery of other beneficial lung agents such as bronchodilators and even intrapulmonary antibiotics.

Application of any therapeutic agent (intrapulmonary, intravascular, etc.) may result in changes in lung compliance, central hemodynamics, or both that then require dynamic and automatic altering and recalibrating of the PPV and NPV parameters that continue optimization of the patients respiratory, cardiovascular, and cerebrovascular physiology.

Also as indicated the use of other external negative or positive pressure producing devices may be used as adjuncts to improve lung function, reduce lung injury associated with PPV, enhance central and peripheral hemodynamics and organ function, improve cerebral perfusion pressure, improve delivery of therapeutic agents to the lung given via inhalation methods, and assist in weaning from mechanical ventilation.

The intelligent control systems 10, 110 described above include three major components:

I. A signal processing component to filter, estimate, predict and transform the measured signals physiological and extract features from them. This can be realized using a combination of a variety of methods including discrete wavelet transform (DWT), discrete Fourier transform (DFT), and discrete cosine transform (DCT).

II. A machine learning component to analyze extracted features and predict when to stop, resume or start ventilation at each specific mode of operation. This can be implemented using Random Forest, support vector machines (SVM), neutral networks (NN), deep learning, error-correcting output codes (ECOC), and other machine learning and artificial intelligence techniques.

III. A Feedback control mechanism to create the optimal levels and frequencies for PEEP and NEEP. This can be implemented using one or more of PID control, model-based control, neural control, optimal control, neuro-fuzzy control, and robust control.

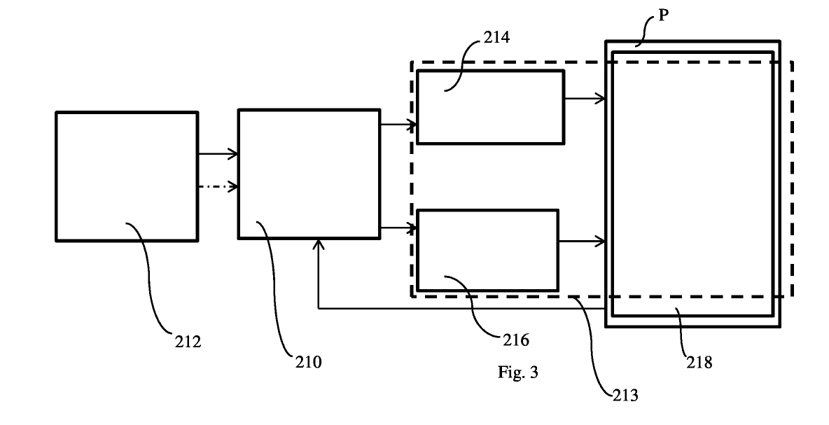

Turning to FIG. 3, a schematic diagram of a third embodiment of a control system 210 of the present disclosure is illustrated, in which external pressure oscillatory devices such as a neck collar or abdominal curiass 213 (illustrated in broken lines) is used either alone or in conjunction with positive pressure and negative pressure ventilation 214, 216 to further effect and optimized ventilatory, hemodynamics, or vital organ perfusion response. As in the first and second embodiments, one or more physiologic measuring devices 218 is provided on the patient, which communicates physiological signals to the controller 210. The controller 210 performs a comparison of the physiological signals to a desired or target value for the physiological parameter associated with that that one or more physiological signal from the memory 212. The memory 212 may either integral with, or as indicated by dash-dotted line, remote from, the control system 210.

While various embodiments have been disclosed, it will be appreciated that variations may be made thereto that are still considered within the scope of the appended claims.

1. A system for controlling at least one of patient ventilation, central hemodynamics, and organ perfusion, comprising:
a controller;
a memory in communication with the controller, the memory including a target value for one or more physiological parameters;
one or more physiological parameter sensors in communication with the controller and a patient; and
a negative pressure ventilation (NPV) and negative end expiratory pressure ventilation (NEEP) system (NPV/NEEP system) in communication with the controller, the controller including a programmed logic control that, based on a comparison of signals received from each of the one or more physiological parameter sensors to a respective one of the target values included in the memory, generates one of a recommendation that an adjustment should be made to negative pressure provided by the NPV/NEEP system, or a signal to the NPVNEEP system to make an adjustment to negative pressure provided by the NVP/NEEP system.

2. The system of claim 1, further comprising a positive pressure ventilation (PPV) and positive end expiratory pressure ventilation (PEEP) system (PPV/PEEP system) in communication with the controller, the programmed logic control of the controller, based on the comparison of signals received from each of the one or more physiological parameter sensors to a respective one of the target values included in the memory, further generating one of a recommendation that an adjustment should be made to positive pressure provided by the PPV/PEEP system, or a signal to the PPV/PEEP system to make an adjustment to positive pressure provided by the PPV/PEEP system.

3. The system of claim 2, wherein, based on the comparison of signals received from each of the one or more physiological parameter sensors to a respective one of the target values included in the memory, the controller generates one of a recommendation that no positive pressure be provided by the PPV/PEEP system, or a signal to the PPV/PEEP system not to deliver positive pressure.

4. The system of claim 2, and based on the comparison of signals received from each of the one or more physiological parameter sensors to a respective one of the target values included in the memory, the controller generates one of a recommendation that the NEEP should be set to deliver a constant level of NEEP at a particular value, a recommendation that the NEEP should be set to deliver a constant level of NEEP within a particular range of values, or a signal to the NEEP to deliver a constant level of negative pressure.

5. The system of claim 4, and based on the comparison of signals received from each of the one or more physiological parameter sensors to a respective one of the target values included in the memory, the controller further generates one of a recommendation that the PEEP be set to deliver positive pressure at a particular frequency and one of at a particular level of positive pressure or within a range of levels of positive pressure, or one or more signals to the PEEP to deliver positive pressure at a particular frequency and a particular level.

6. The system of claim 1, and based on the comparison of signals received from each of the one or more physiological parameter sensors to a respective one of the target values included in the memory, the controller generates one of a recommendation that the NEEP should be set to deliver a constant level of NEEP at a particular value, a recommendation that the NEEP should be set to deliver a constant level of NEEP within a particular range of values, or a signal to the NEEP to deliver a constant level of negative pressure.

7. The system of claim 1, the intelligent control system comprising:
a signal processing component to filter, estimate, predict and transform the measured signals physiological and extract features from them;
a machine learning component to analyze extracted features and predict when to stop, resume or start ventilation at each specific mode of operation; and. This can be implemented using Random Forest, support vector machines (SVM), neutral network (NN), error-correcting output codes (ECOC), and other machine learning and artificial intelligence techniques; and
a feedback control mechanism to create the optimal levels and frequencies for PEEP and NEEP.

8. The system of claim 7, the signal processing component using a combination of two or more of a group including discrete wavelet transform (DWT), discrete Fourier transform (DFT), and discrete cosine transform (DCT).

9. The system of claim 7, the machine learning component using at least one of Random Forest, support vector machines (SVM), neutral network (NN), error-correcting output codes (ECOC), and other machine learning and artificial intelligence techniques.

10. The system of claim 7, the feedback control mechanism using one or more of PID control, model-based control, optimal control, neuro-fuzzy control, neural control, and robust control.

11. The system of claim 1, further comprising a display in communication with the controller, the display displaying messages corresponding to recommendations generated by the controller.

12. The system of claim 1, further comprising an external pressure oscillatory device.

13. The system of claim 12, wherein the external pressure oscillatory device comprises at least one of a group of a neck collar and an abdominal curiass.

14. The system of claim 2, further comprising an external pressure oscillatory device.

15. The system of claim 14, wherein the external pressure oscillatory device comprises at least one of a group of a neck collar and an abdominal curiass.

16. The system of claim 1, wherein the one or more physiological parameter sensors are sensors to measure one or more of the group including: esophageal pressure, intraabdominal pressure, bladder pressure, arterial blood pressures, central venous pressure, pulmonary artery pressure, arterial oxygenation, venous oxygenation, tissue oxygenation, electric activity of the heart, impedance, cerebral blood flow, cardiac output, stroke volume variation, and pulse pressure variation.

17. The system of claim 4, and based on the comparison of signals received from each of the one or more physiological parameter sensors to a respective one of the target values included in the memory, the controller generates one of a recommendation that the NPV should be set to deliver an oscillatory magnitude of negative pressures and a recommendation that the NPV should be set to deliver an oscillatory frequency of negative pressures.

18. A system for optimizing the delivery of intrapulmonary therapeutic agents including at least one of surfactants, bronchodilators, mucoytics, and antibiotics, comprising:
a controller;
a memory in communication with the controller, the memory including a target value for one or more physiological parameters;
one or more physiological parameter sensors in communication with the controller and a patient; and
a negative pressure ventilation (NPV) and negative end expiratory pressure ventilation (NEEP) system (NPV/NEEP system) in communication with the controller, the controller including a programmed logic control that, based on a comparison of signals received from each of the one or more physiological parameter sensors to a respective one of the target values included in the memory, generates one of a recommendation that an adjustment should be made to negative pressure provided by the NPV/NEEP system, or a signal to the NPVNEEP system to make an adjustment to negative pressure provided by the NVP/NEEP system.

19. A method of controlling the level of intracranial pressure (ICP) in a patient, comprising:
measuring ICP of the patient;
providing signals indicative of the measured ICP to an intelligent control system;
performing, via the intelligent control system, a comparison of values associated with the signals to a respective desired or target value for the ICP from a memory; and
sending, via the intelligent control system, control signals to a negative end expiratory pressure ventilation (NEEP) system based on the comparison, or sending a recommendation that an adjustment be made, or no adjustment be made, to a particular level and/or frequency of, the negative pressure delivered by the NEEP.

20. The method of claim 19, further comprising measuring at least one additional physiologic parameter including at least one of a group including arterial blood pressure (ABP) and cerebral pressure perfusion, and providing signals indicative of the measured at least one additional physiologic parameter to the intelligent control system, and the comparison of values associated with the signals to a respective desired or target value for the ICP from a memory includes a comparison of values associated with the at least one additional physiologic parameter to a respective desired or target value for the additional physiologic parameter.

21. The method of claim 19, further comprising sending, via the intelligent control system, control signals to a positive end expiratory pressure ventilation (PEEP) system based on the comparison, or sending a recommendation that an adjustment be made, or no adjustment be made, to a particular level and/or frequency of, the negative pressure delivered by the PEEP.

22. A method of controlling the level of at least one physiologic parameter including cardiac output, tissue oxygenation, and lung pressures in a patient, comprising:
measuring physiologic parameters including at least one of cardiac output, tissue oxygenation, and lung pressures of the patient;
providing signals indicative of the measured physiologic parameters to an intelligent control system;
performing, via the intelligent control system, a comparison of values associated with the signals to a respective desired or target value for the physiologic parameters from a memory; and
sending, via the intelligent control system, control signals to a negative end expiratory pressure ventilation (NEEP) system based on the comparison, or sending a recommendation that an adjustment be made, or no adjustment be made, to a particular level and/or frequency of, the negative pressure delivered by the NEEP.

23. The method of claim 22, further comprising measuring at least one additional physiologic parameter including at least one of a group including arterial blood pressure (ABP) and cerebral pressure perfusion, and providing signals indicative of the measured at least one additional physiologic parameter to the intelligent control system, and the comparison of values associated with the signals to a respective desired or target value for the physiologic parameter including cardiac output, tissue oxygenation, or lung pressure from a memory includes a comparison of values associated with the at least one additional physiologic parameter to a respective desired or target value for the additional physiologic parameter.

24. The method of claim 22, further comprising sending, via the intelligent control system, control signals to a positive end expiratory pressure ventilation (PEEP) system based on the comparison, or sending a recommendation that an adjustment be made, or no adjustment be made, to a particular level and/or frequency of, the negative pressure delivered by the PEEP.
